(19) United States
(12) Patent Application Publication
(10) Pub. No.: US 2016/0186081 A1
(43) Pub. Date: Jun. 30, 2016

(54) ENVIRONMENTAL PROTECTION LIQUID FUEL GENERATOR

(71) Applicants: Chih-Jung CHAO, New Taipei City (TW); Lian-Nak TI, SUBANG JAYA (MY)

(72) Inventors: Chih-Jung CHAO, New Taipei City (TW); Lian-Nak TI, SUBANG JAYA (MY)

(21) Appl. No.: 14/586,223

(22) Filed: Dec. 30, 2014

(51) Int. Cl. C10L 1/02; C11C 3/04; C12P 7/64; B01D 3/00; C12M 1/00

(52) U.S. Cl. CPC C10L 1/026; B01D 3/009; C12M 21/12; C12P 7/649; C11C 3/04; C10L 2270/026; C10L 2200/0476; C10L 2290/543; C10L 2290/26

(57) ABSTRACT

An environmental protection liquid fuel generator, in which the fuel oil generator is a structure made from a receiving device, an esterification device, a first standing separation device, a compression distillation device, an acid-base neutralization device, a second standing separation device, and a decompression distillation device. A mixed proportion of a vegetable oil and an alkide (catalyzing enzyme) is placed into the fuel generator, and the esterification device is used to increase electron affinity and accelerate the reaction rate, thereby enabling the distillation of low polluting and low sulfur containing environmental protection fuel oil.

### BACKGROUND OF THE INVENTION

(a) Field of the Invention

The present invention provides an environmental protection liquid fuel generator, which places a mixed proportion of a vegetable oil and an alkide (catalyzing enzyme) into a fuel generator, and uses an esterification device to increase electron affinity and accelerate the reaction rate, thereby enabling distillation of low polluting and low sulfur containing environmental protection fuel oil.

(b) Description of the Prior Art

Rapid industrial development and the increase in use of vehicles have brought about the serious problems of air pollution, and has become an important issue that everyone needs to squarely face up to. The components of the exhaust gas emitted after fuel combustion pollute the air and affect human health, as well as causing adverse effects on the earth from gradual global warming. Referring to FIG. 1, which shows the ecological cycle of current oil material and includes a. planting, growing, and harvesting of crops; b. processing of crops to manufacture oil material; c. subpackaging and transporting of the oil material; d. fuel burning engines using the oil material; and e. mixing of the gas emission with air and light. Accordingly, air quality becomes progressively worse through the effects of this cyclic production, which impacts crop development and nourishment. Hence, the standards for material emissions into the air need to be lowered to achieve an improvement in air pollution.

An essential factor in improving air pollution and emission standards is the lowering of the sulfur content in fuel oil, with an objective of implementing a plan to lower the sulfur content of petroleum products to 0.3%. However, there are limitations to lowering sulfur content, as well as limitations on the production capacity of low sulfur fuel oil and difficulties in purchase thereof.

Nevertheless, there are two approaches that can be taken to resolve and improve the existing shortcomings and problems derived from using diesel oil, including using biological diesel oil (BIO Diesel) and dimethyl ether (DME). These two materials are both environmental protection materials obtained by extraction and purification, and can be used as a substitute for diesel oil, thereby reducing pollution problems produced by existing petroleum diesel oil. Furthermore, these two products were developed because of their environmental protection qualities. However, they are not the optimum substitutes and have the following shortcomings: BIO Diesel: 1. cold starting of a vehicle is poor; 2. excessive high speed operating horsepower, and poor high speed performance; and dimethyl ether (DME): 1. Unsafe—DME is a toxic gaseous fuel, which very easily results in gas leakage, bringing about intoxication; 2. high cost; 3. poor lubricity—during fuel injection the DME has no lubricity and thus requires the addition of a lubricating oil to increase the degree of lubrication of the fuel. Moreover, currently developed BIO Diesel oils still rely on the refinement of mineral diesel oil, and when using a BIO Diesel oil containing a high content of mineral diesel oil pollution problems similar to using petroleum diesel oil still exist. In addition, the same hidden pollution problems still exist because of the air pollution component sulfur contained in mineral diesel oil.

Hence, in light of the shortage of energy resources and the problems of environment pollution brought about by using energy resources, the author of the present invention has attentively researched various methods to resolve such issues to finally develop an environmental protection liquid fuel generator. The environmental protection liquid fuel generator uses an alkide (catalyzing enzyme) and a vegetable oil, and adopts an esterification device of a fuel oil generator to increase electron affinity and accelerate the reaction rate to produce an environmental protection synthetic low sulfur fuel oil. Accordingly, the present invention achieves a fuel oil that is more environmentally friendly, as well as being efficient, safe, and of low cost. In addition, when used in combination with BIO Diesel oil, general top grade diesel oil or common diesel oil, the environmental protection synthetic low sulfur fuel oil has the ability to resolve or alleviate the problems of conventional high sulfur diesel oil.

### SUMMARY OF THE INVENTION

The primary objective of the present invention is to provide an environmental protection liquid fuel generator, wherein an embodiment of the liquid fuel generator comprises a receiving device, an esterification device, a first standing separation device, a compression distillation device, an acid-base neutralization device, a second standing separation device, and a decompression distillation device.

A second objective of the present invention is to provide the environmental protection liquid fuel generator wherein a mixed proportion of a vegetable oil and an alkide is placed in an embodiment of the liquid fuel generator, and the esterification device is used to bring about a reaction and the distillation of low polluting and low sulfur containing environmental protection fuel oil.

To enable a further understanding of said objectives and the technological methods of the invention herein, a brief description of the drawings is provided below followed by a detailed description of the preferred embodiments.

### BRIEF DESCRIPTION OF THE DRAWINGS

FIG. 1 is a schematic view of an ecological cycle of oil material of the prior art.

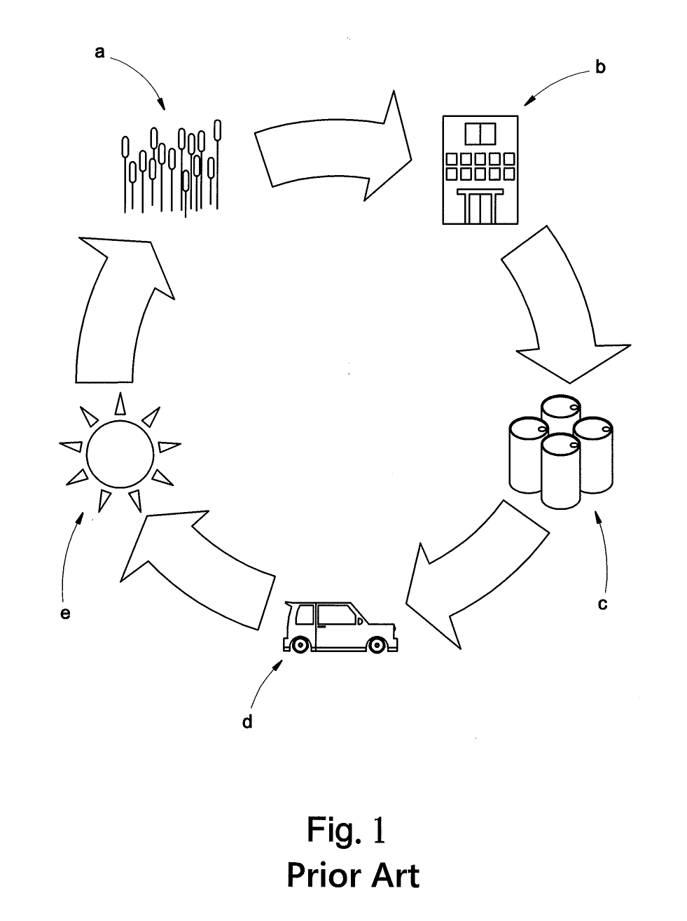

FIG. 2 is a schematic view of a fuel oil generator for an environmental protection liquid fuel oil generator of the present invention.

FIG. 3 is a schematic flow chart of material addition for an environmental protection liquid fuel oil generator of the present invention.

### DETAILED DESCRIPTION OF THE PREFERRED EMBODIMENTS

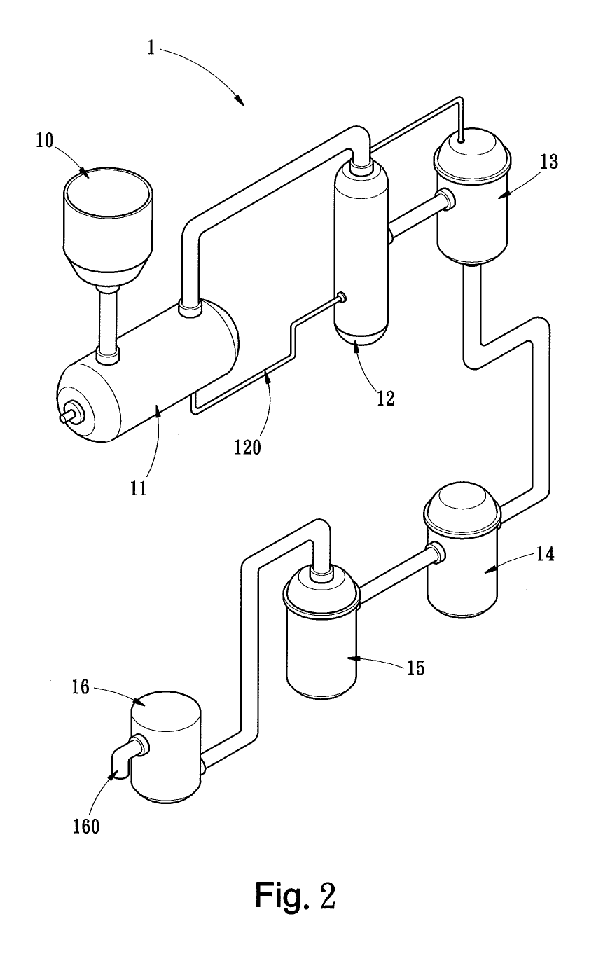

Referring first to FIG. 2, which shows an environmental protection liquid fuel generator of the present invention that is a synthetic low sulfur fuel oil generator, wherein a fuel oil generator 1 is a structure comprising: a receiving device 10, an esterification device 11, a first standing separation device 12, a compression distillation device 13, an acid-base neutralization device 14, a second standing separation device 15, and a decompression distillation device 16, The receiving device 10 is a feeder container, and the receiving device corresponds to the esterification device 11. The esterification device 11 enables heating of the additives, which respectively corresponds to the receiving device 10 and the first standing separation device 12. The first standing separation device 12 enables separation of ester oil and glycerine, which respectively corresponds to the esterification device 11 and the compression distillation device 13. Moreover, the first standing separation device 12 is provided with a gas recovery conduit 120 to be corresponded to the esterification device 11. The compression distillation device 13 enables removal of water content, which respectively corresponds to the first standing separation device 12 and the acid-base neutralization device 14. The acid-base neutralization device 14 enables the addition of acetic acid to the material mixture, which respectively corresponds to the compression distillation device 13 and the second standing separation device 15. The second standing separation device 15 enables separation of ester oil and water content, which respectively corresponds to the acid-base neutralization device 14 and the decompression distillation device 16. The decompression distillation device 16 enables refinement of the environmental protection liquid fuel oil, with the refined fuel oil being extracted from an extraction outlet 160. Accordingly, an environmental protection liquid fuel oil for use is extracted and purified by means of the fuel oil generator 1.

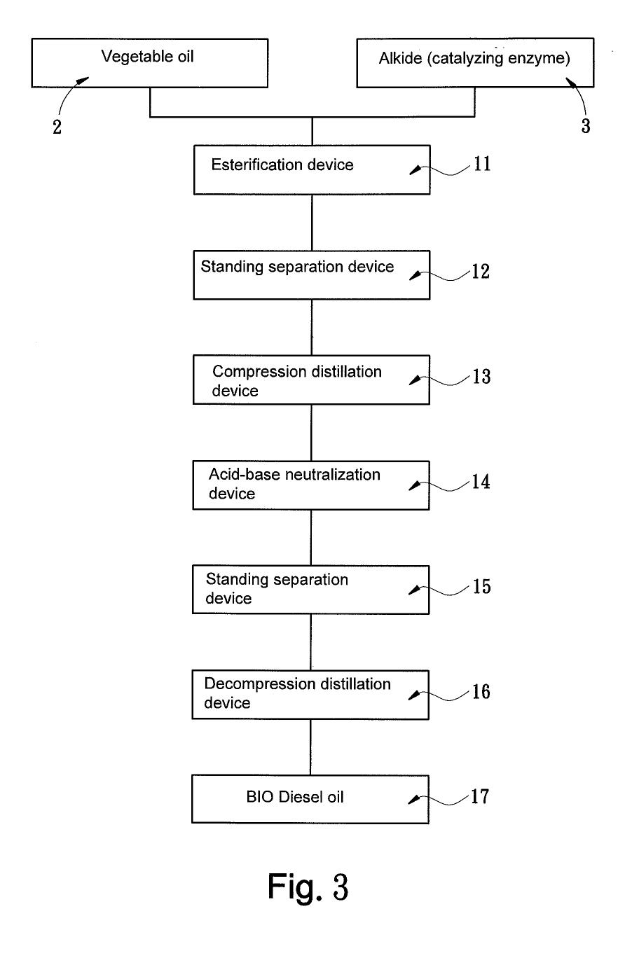

Referring next to FIG. 2 and FIG. 3, which show the environmental protection liquid fuel generator of the present invention, wherein fuel oil with environmental protection characteristics produced by the fuel oil generator 1 forms a synthetic low sulfur biological diesel oil (BIO Diesel) 17. The BIO Diesel oil 17 with low sulfur content is primarily formed by adding a mixed proportion of a vegetable oil and an alkide (catalyzing enzyme) to the fuel oil generator 1, which extracts and purifies the BIO Diesel oil to reduce the amount of sulfur contained in the fuel oil and provide for different flash points.

Accordingly, the mixed proportion of an alkide (catalyzing enzyme), which is a decane, an 11 alkane, or a 12 alkane, with a vegetable oil required a flash point between 45° C.˜70.5° C., and the sulfur content was below 2.3 ppm (parts per million). Hence, with a sulfur content below 2.3 ppm, it produces a gas that is close to being completely non-polluting. Moreover, after use, the discharged waste gas is environmentally friendly and non-polluting, and no black smoke is produced.

Referring to the test results from a SGS Test Report, wherein proportion by weight of a vegetable oil and alkide (catalyzing enzyme) is 50%+50%, and the vegetable oil is:

a. soybean oil with flash point: 45° C., and sulfur content: 0.22 ppm;

Test Results:

| Test Item | Unit | Test Method | Test Result |
|---|---|---|---|
| 1. Density at 15.5° C. | g/ml | ASTM D4052 | 0.8496 |
| 2. Flash point | ° C. | ASTM D93 | 45.0 |
| 3. Ignition point | ° C. | ASTM D92 | 56 |
| 4. Water content & sediment | Vol % | ASTM D1796 | <0.1 |
| 5. Viscosity at 40° C. | cSt | ASTM D445 | 4.538 |
| 6. Ash mass content | wt % | ASTM D482 | <0.01 |
| 7. Sulfur content | ppm | ASTM D5453 | 0.22 |
| 8. Copper corrosiveness | — | ASTM D130 | 1a |
| 9. Pour point | ° C. | ASTM D97 | −30 |
| 10. Carbon residue | wt % | ASTM D4530 | 0.18 |
| 11. Gross heat of combustion | Kcal/Kg | ASTM D240 | 10254 |
| 12. Net heat of combustion | Kcal/Kg | ASTM D240 | 9720 |
| 13. Carbon content | wt % | ASTM D5291 | 72.93 |
| 14. Hydrogen content | wt % | ASTM D5291 | 10.54 |
| 15. Oxygen content | wt % | Element | 6.65 |
| | | analyzer | |
| 16. Phosphorus content | ppm | Element | Not detected |
| | | analyzer | (<2) |

1a: Light orange color, almost the same color as freshly polished copper strip

b. cotton seed oil with flash point: 45° C., sulfur content: 1.1 ppm;

Test Results:

| Test Item | Unit | Test Method | Test Result |
|---|---|---|---|
| 1. Density at 15.5° C. | g/ml | ASTM D4052 | 0.8492 |
| 2. Flash point | ° C. | ASTM D93 | 45.0 |
| 3. Ignition point | ° C. | ASTM D92 | 56 |
| 4. Water content & sediment | Vol % | ASTM D1796 | <0.1 |
| 5. Viscosity at 40° C. | cSt | ASTM D445 | 4.492 |
| 6. Ash mass content | wt % | ASTM D482 | <0.01 |
| 7. Sulfur content | ppm | ASTM D5453 | 1.1 |
| 8. Copper corrosiveness | — | ASTM D130 | 1a |
| 9. Pour point | ° C. | ASTM D97 | −27 |
| 10. Carbon residue | wt % | ASTM D4530 | 0.21 |
| 11. Gross heat of combustion | Kcal/Kg | ASTM D240 | 10233 |
| 12. Net heat of combustion | Kcal/Kg | ASTM D240 | 9699 |
| 13. Carbon content | wt % | ASTM D5291 | 79.59 |
| 14. Hydrogen content | wt % | ASTM D5291 | 10.54 |
| 15. Oxygen content | wt % | Element | 4.52 |
| | | analyzer | |
| 16. Phosphorus content | ppm | Element | Not detected |
| | | analyzer | (<2) |

1a: Light orange color, almost the same color as freshly polished copper strip

c. rapeseed oil with flash point: 45° C., sulfur content: 2.3 ppm;

Test Results:

| Test Item | Unit | Test Method | Test Result |
|---|---|---|---|
| 1. Density at 15.5° C. | g/ml | ASTM D4052 | 0.8477 |
| 2. Flash point | ° C. | ASTM D93 | 45.0 |
| 3. Ignition point | ° C. | ASTM D92 | 56 |
| 4. Water content & sediment | Vol % | ASTM D1796 | <0.1 |
| 5. Viscosity at 40° C. | cSt | ASTM D445 | 4.614 |
| 6. Ash mass content | wt % | ASTM D482 | <0.01 |
| 7. Sulfur content | ppm | ASTM D5453 | 2.3 |
| 8. Copper corrosiveness | — | ASTM D130 | 1a |
| 9. Pour point | ° C. | ASTM D97 | −39 |
| 10. Carbon residue | wt % | ASTM D4530 | 0.21 |
| 11. Gross heat of combustion | Kcal/Kg | ASTM D240 | 10230 |
| 12. Net heat of combustion | Kcal/Kg | ASTM D240 | 9732 |
| 13. Carbon content | wt % | ASTM D5291 | 68.99 |
| 14. Hydrogen content | wt % | ASTM D5291 | 9.83 |
| 15. Oxygen content | wt % | Element | 4.98 |
| | | analyzer | |
| 16. Phosphorus content | ppm | Element | Not detected |
| | | analyzer | (<2) |

1a: Light orange color, almost the same color as freshly polished copper strip

d. palm oil with flash point: 45° C., sulfur content: 2.3 ppm.

Test Results:

| Test Item | Unit | Test Method | Test Result |
|---|---|---|---|
| 1. Density at 15.5° C. | g/ml | ASTM D4052 | 0.8445 |
| 2. Flash point | ° C. | ASTM D93 | 45.0 |
| 3. Ignition point | ° C. | ASTM D92 | 56 |
| 4. Water content & sediment | Vol % | ASTM D1796 | <0.1 |
| 5. Viscosity at 40° C. | cSt | ASTM D445 | 4.371 |
| 6. Ash mass content | wt % | ASTM D482 | <0.01 |
| 7. Sulfur content | ppm | ASTM D5453 | 1.1 |
| 8. Copper corrosiveness | — | ASTM D130 | 1a |
| 9. Pour point | ° C. | ASTM D97 | −15 |
| 10. Carbon residue | wt % | ASTM D4530 | 0.23 |
| 11. Gross heat of combustion | Kcal/Kg | ASTM D240 | 10276 |
| 12. Net heat of combustion | Kcal/Kg | ASTM D240 | 9739 |
| 13. Carbon content | wt % | ASTM D5291 | 78.64 |
| 14. Hydrogen content | wt % | ASTM D5291 | 10.59 |
| 15. Oxygen content | wt % | Element | 3.95 |
| | | analyzer | |
| 16. Phosphorus content | ppm | Element | Not detected |
| | | analyzer | (<2) |

1a: Light orange color, almost the same color as freshly polished copper strip

In the test results, when the flash point is at 45° C., the sulfur content is within 2.3 ppm, which clearly achieves a reduction in the discharge amount of sulfur.

When the proportion by weight of a vegetable oil and alkide (catalyzing enzyme) is 75%+25%, the test results were as follows:

Test Results:

| Test Item | Unit | Test Method | Test Result |
|---|---|---|---|
| 1. Density at 15.5° C. | g/ml | ASTM D4052 | 0.8834 |
| 2. Flash point | ° C. | ASTM D93 | 51.0 |
| 3. Viscosity at 50° C. | cSt | ASTM D445 | 8.210 |
| 4. Viscosity at 70° C. | cSt | ASTM D445 | 5.438 |
| 5. Viscosity at 90° C. | cSt | ASTM D445 | 4.044 |
| 6. Viscosity at 100° C. | cSt | ASTM D445 | 3.512 |
| 7. Ash mass content | wt % | ASTM D482 | 0.003 |
| 8. Sulfur content | ppm | ASTM D5433 | 2.2 |
| 9. Pour point | ° C. | ASTM D97 | −27 |
| 10. Gross heat of combustion | Kcal/Kg | ASTM D240 | 9905 |
| 11. Net heat of combustion | Kcal/Kg | ASTM D240 | 9180 |
| 12. Carbon content | wt % | Element | 72.50 |
| | | analyzer | |
| 13. Hydrogen content | wt % | Element | 14.3 |
| | | analyzer | |
| 14. Oxygen content | wt % | Element | 8.72 |
| | | analyzer | |

When the flash point is 51° C., the sulfur content is within 2.2 ppm. Hence, the values produced by different mixed proportions of the vegetable oil and alkide similarly achieve a sulfur content lower than 2.3 ppm.

When the proportion by weight of a vegetable oil and alkide (catalyzing enzyme) is 60%+40%, the test results were as follows:

Test Results:

| Test Item | Unit | Test Method | Test Result |
|---|---|---|---|
| 1. Density at 15.5° C. | g/ml | ASTM D4052 | 0.9502 |
| 2. Flash point | ° C. | ASTM D93 | 95.0 |
| 3. Viscosity at 50° C. | cSt | ASTM D445 | 7.137 |
| 4. Viscosity at 70° C. | cSt | ASTM D445 | 4.731 |
| 5. Viscosity at 90° C. | cSt | ASTM D445 | 3.368 |
| 6. Viscosity at 100° C. | cSt | ASTM D445 | 2.966 |
| 7. Ash mass content | wt % | ASTM D482 | <0.001 |
| 8. Sulfur content | ppm | ASTM D2622 | 0.006 |
| 9. Pour point | ° C. | ASTM D97 | <−45 |
| 10. Gross heat of combustion | Kcal/Kg | ASTM D240 | 9721 |
| 11. Net heat of combustion | Kcal/Kg | ASTM D240 | 9192 |
| 12. Carbon content | wt % | Element | 81.67 |
| | | analyzer | |
| 13. Hydrogen content | wt % | Element | 10.43 |
| | | analyzer | |
| 14. Oxygen content | wt % | Element | 5.62 |
| | | analyzer | |

When the flash point is 95° C., the sulfur content is within 0.006 ppm. Accordingly, the higher the flash point, the lower the sulfur content produced. In addition, the discharged gas is non-polluting and no black smoke is produced. Hence, there is no production of acid rain to break down the atmospheric layer, thus, global warming can be slowed down to alleviate changes in the global climate system and the ecological environment.

When the proportion by weight of a vegetable oil and an alkide (catalyzing enzyme) is 50%+50%, then the cetane number test results for the respective vegetable oils were:

a. palm oil→45

Test Results:

| Test Item | Unit | Test Method | Test Result |
|---|---|---|---|
| Cetane number | — | ASTM D613 | 45.0 |

b. colza oil→43.4

Test Results:

| Test Item | Unit | Test Method | Test Result |
|---|---|---|---|
| Cetane number | — | ASTM D613 | 43.4 |

c. cotton seed oil→43.4

Test Results:

| Test Item | Unit | Test Method | Test Result |
|---|---|---|---|
| Cetane number | — | ASTM D613 | 43.4 |

d. soybean oil→43.7

Test Results:

| Test Item | Unit | Test Method | Test Result |
|---|---|---|---|
| Cetane number | — | ASTM D613 | 43.7 |

In the test results, the cetane numbers averaged between 43.4˜45, thus effectively improving the starting capability of diesel oil engines, decreasing the emission of NOX (nitrogen oxide), CO (carbon monoxide), and THC (Total Hydrocarbons), as well as reducing fuel consumption and combustion noise.

Based on the test results as described above, the derived advantages of the present invention are as follows:

1. Able to substantially decrease the emission of black smoke and discharge and dispersal of nitrides, as well as being fuel efficient.

2. Combustion explosiveness is higher and stable.

3. 100% compatible for direct use in existing diesel engines.

4. No need to modify the engine system when mixed 50% with general diesel oil or common diesel oil, and improves engine efficiency.

Furthermore, when the vegetable oil and the alkide (catalyzing enzyme) are proportionally mixed, the higher the flash point, the lower the sulfur content, achieving almost zero sulfur content. And when the flash point is slightly lower, the sulfur content is similarly within a safety range, again achieving almost zero sulfur content. The value of the flash point can be adjusted according to customer requirements to achieve a value within a preferred range.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. An environmental protection liquid fuel generator, comprising a receiving device, an esterification device, a first standing separation device, a compression distillation device, an acid-base neutralization device, a second standing separation device, and a decompression distillation device; the esterification device is used to increase electron affinity of the additives and accelerate the reaction rate to distill low polluting and low sulfur containing environmental protection fuel oil.

2. A manufacturing process of an environmental protection liquid fuel oil, wherein the process comprises a mixture of a vegetable oil and a catalyzing enzyme, and a mixed proportion of the vegetable oil and the alkide (catalyzing enzyme) is placed in a fuel oil generator; wherein the required flash point of a mixed proportion of the alkide (catalyzing enzyme), which is a decane, an 11 alkane, or a 12 alkane, and the vegetable oil is between 45° C.˜70.5° C., and the sulfur content is below 2.3 ppm (parts per million); wherein, with a sulfur content below 2.3 ppm, after use, discharged waste gas produced by the sulfur is environmentally friendly and non-polluting, and no black smoke is produced.

3. The manufacturing process of the environmental protection liquid fuel oil according to claim 2, wherein the process includes a mixture of a vegetable oil and a catalyzing enzyme, wherein:
a preferred proportion by weight of a vegetable oil and the catalyzing enzyme is 60%+40% respectively, which has a flash point of 95° C. that is able to reduce the sulfur content to 0.006 ppm;
a preferred proportion by weight of a vegetable oil and the catalyzing enzyme is 75%+25% respectively, which has a flash point of 51° C. that is able to reduce the sulfur content to 2.2 ppm;
a preferred proportion by weight of a vegetable oil and the catalyzing enzyme is 50%+50% respectively, which has a flash point of 45° C. that is able to reduce the sulfur content to below 2.3 ppm;
a preferred proportion by weight of a vegetable oil and the catalyzing enzyme is 50%+50% respectively, having cetane number between 43.4˜45.

4. The environmental protection synthetic low sulfur fuel oil according to claim 2, wherein the vegetable oil used is palm oil, colza oil, cotton seed oil, or soybean oil.
